(19) United States
(12) Patent Application Publication
(10) Pub. No.: US 2012/0010552 A1
(43) Pub. Date: Jan. 12, 2012

(54) DISPOSABLE APPLICATOR ASSEMBLY

(75) Inventors: Les Greer, JR., Sandpoint, ID (US); Marcus Anderson, Newport, WA (US)

(73) Assignee: Unicep Packaging, Inc., Sandpoint, ID (US)

(21) Appl. No.: 12/831,782

(22) Filed: Jul. 7, 2010

(51) Int. Cl. A61M 35/00; B23P 17/04; A61B 19/02

(52) U.S. Cl. 604/1; 206/438; 29/428

(57) ABSTRACT

A disposable applicator assembly includes a handle of a diameter substantially larger than the diameter of an applicator stick. The applicator assembly includes a casing, the applicator stick, and an applicator (e.g., a swab). A support structure is disposed within the casing and engages the applicator stick. A product may be housed in the casing for delivery by the applicator. The applicator assembly may be formed of a single material that is suitable for an injection molding process.

### BACKGROUND

Swab assemblies exist to deliver a pre-proportioned product in a disposable and sterile manner. Such devices usually consist of a stick with a swab disposed at one end either immersed or covered in a product enclosed in a casing. Swab assemblies can make application of cosmetic and oral products much easier, as the product is pre-proportioned, the swab provides an extended reach, the swab end is sterile, and the swab end provides a soft applicator for sensitive areas of the mouth or face.

Swab assemblies have generally been designed with a handle portion for the user to grasp in order to use the swab contained inside. Typically, the handle portion of the swab assembly has a substantially similar diameter to the diameter of the swab stick. However, for many swab assembly uses this relatively small handle diameter may prove difficult to use because of the smaller surface area that the user is afforded to grasp.

### SUMMARY

This summary is provided to introduce simplified concepts of disposable applicator assemblies. Additional details of example disposable applicator assemblies are further described below in the Detailed Description. This summary is not intended to identify essential features of the claimed subject matter, nor is it intended for use in determining the scope of the claimed subject matter.

This disclosure is directed to disposable applicator assemblies with handles of a diameter substantially larger than a diameter of an applicator stick. Generally, the disposable applicator assembly includes a casing, a stick, and an applicator (e.g., a swab). A support structure is coupled to the assembly and engages the stick. A product may be housed in the casing for delivery by the applicator.

In one example, the casing has a first hollow portion having an inside diameter and a second hollow portion having an inside diameter substantially the same as the inside diameter of the first hollow portion. The stick is contained within the first and second hollow portions of the casing. An applicator is disposed at one end of the stick. In this example, the support structure is coupled to the casing and engages the stick to hold the stick in place within the casing.

In another example, an elongated swab applicator may have a swab attached at one end. A stick end of the swab applicator is coupled to a support structure, which is interposed between a wall of a casing and the stick end of the elongated swab applicator.

In yet another example, the disposable applicator assembly may be made by forming a casing, forming a support structure coupled to the casing, and inserting a applicator into the support structure.

### BRIEF DESCRIPTION OF THE DRAWINGS

The detailed description is set forth with reference to the accompanying figures. In the figures, the left-most digit(s) of a reference number identifies the figure in which the reference number first appears. The use of the same reference numbers in different figures indicates similar or identical items.

FIG. 1 illustrates an example disposable applicator assembly (in this case a swab assembly) comprising a casing, a stick housed in the casing, and a swab disposed on an end of the stick.

FIG. 2 illustrates an example embodiment of an end configuration and of a failure zone of the swab assembly of FIG. 1.

FIGS. 3A and 3B illustrate other example embodiments of an end configuration and a failure zone of a swab assembly.

FIG. 4 illustrates an example embodiment of a support structure.

FIG. 5 illustrates a further example embodiment of a support structure.

FIG. 6 illustrates a further example embodiment of a support structure.

FIG. 7 illustrates a further example embodiment of a support structure.

FIG. 8 illustrates a further example embodiment of a support structure.

FIGS. 9A, 9B, 9C, 9D, 9E, 9F, and 9G illustrate other example applicators that may be used with applicator assemblies, such as those described herein.

FIG. 10 illustrates an example process for manufacturing a disposable swab assembly.

### DETAILED DESCRIPTION

### Overview

As discussed above, existing swab assemblies are often difficult to grip. This application describes disposable swab assemblies having a handle to facilitate gripping. Generally, the disposable swab assemblies comprise a hollow casing housing a swab stick, and a support structure coupled to the casing and engaging the stick. In some example disposable swab assemblies, the support structure may provide the handle with a diameter substantially larger than a diameter of a swab stick, while still firmly holding the swab stick in place in the casing. In some examples, the diameter of the handle may be substantially the same as a diameter of the casing housing the swab.

Generally, the support structure comprises one or more structural features interposed between the swab stick and the casing to hold the swab stick in place in the housing and to provide a larger handle for the swab assembly. In one embodiment, for example, the support structure comprises ribs that extend radially from the stick to a wall of the casing and are coupled to the assembly. In another embodiment, the support structure comprises a set of cylinders or generally cylindrical shapes that are coupled to the assembly and give support to the stick. In other implementations, the support structure may comprise a number of cylinders further comprising at least a first cylinder coupled to the inner diameter of the second hollow portion and a second cylinder engaging the stick and coupled to an end of the second hollow portion. In still other embodiments, other shapes, sizes and configurations of support structure may be used.

In some implementations, the disposable swab assembly may be manufactured using an injection molding process. Other manufacturing techniques are also contemplated. For example, a blow-fill-seal process, a blow molding process or any other manufacturing process suitable for forming the dispenser may be used. Depending on the product to be housed in the disposable swab assembly and the manufacturing process, the disposable swab assembly may comprise a polymer, such as polyethylene, ethyl vinyl alcohol copolymer or any other suitable polymer, mixture or the like that is suitable for forming the disposable swab assembly. By way of example and not limitation, low-density polyethylene (LDPE), high-density polyethylene (HDPE) or, polypropylene (PP) may be used to form part or all of the disposable swab assembly.

As used herein, the term “swab” includes not only cotton swabs and synthetic swabs such as those used in the personal care setting, but also other swabs made of organic, inorganic, and/or composite materials (e.g. natural or synthetic sponges, natural or synthetic fibers, plastic fibers, cellulose fibers, or the like). Swabs may include both fibrous and non-fibrous materials. Also, while embodiments are described herein in terms of assemblies housing swab applicators other types of applicators and/or implements may also be used (e.g., brushes, picks, needles, sticks, tongue depressors, pipettes, or the like).

### Illustrative Disposable Swab Assembly

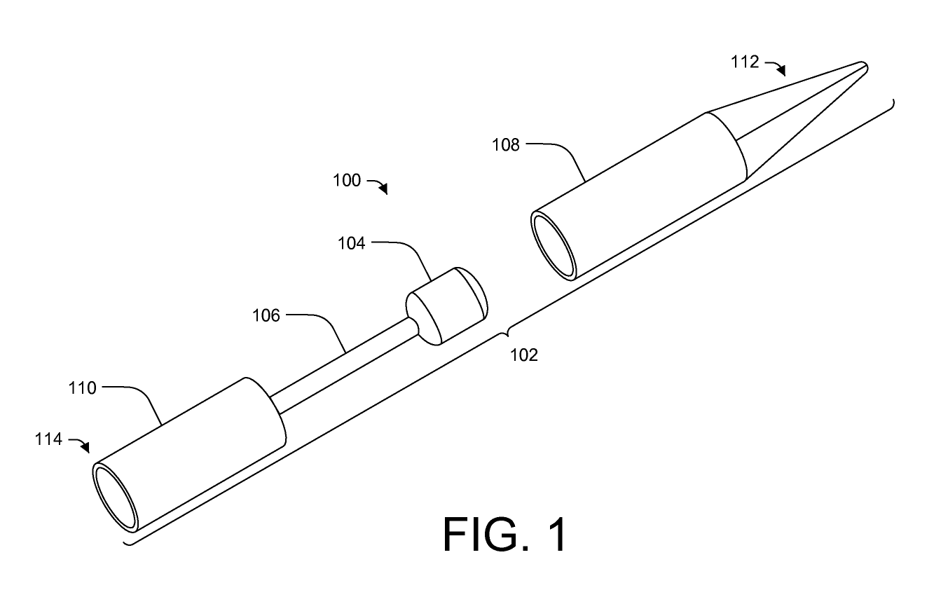

FIG. 1 is a perspective view of an illustrative opened disposable swab assembly 100 comprising a casing 102, a swab 104, and a stick 106. The casing 102 has a wall thickness and comprises a first hollow portion 108 having an inside diameter and a second hollow portion 110 having an inside diameter substantially the same as the inside diameter of the first hollow portion 108. Prior to opening the swab assembly 100, the stick 106 is contained within the first and second hollow portions 108 and 110 of the casing 102, with the swab 104 disposed at one end of the stick 106. In this illustrative view of the opened disposable swab assembly, the first hollow portion 108 of the casing 102 is narrowed toward an end 112 of the first hollow portion 108 in this particular embodiment. However, the end 112 may be crimped in other embodiments depending on the manufacturing process chosen. An end 114 of the second hollow portion 110 may be cylindrically shaped in some embodiments or otherwise shaped by a manufacturing process without being crimped. In some embodiments, end 114 is disposed opposite the end 112.

Furthermore, the casing 102 is a generally cylindrical shape in this embodiment but could alternatively be any shape that has an average diameter that could be generally described as being cylindrical. For example, the casing 102 could have an octagonal, rectangular, or ergonomically-shaped cross-section. The swab 104 may be used to apply any of a number of products. Products may comprise, for example, cosmetics, oral care products, medicinal products, vehicular lubricants, or any other liquid or powder product that may be applied using a swab and stick.

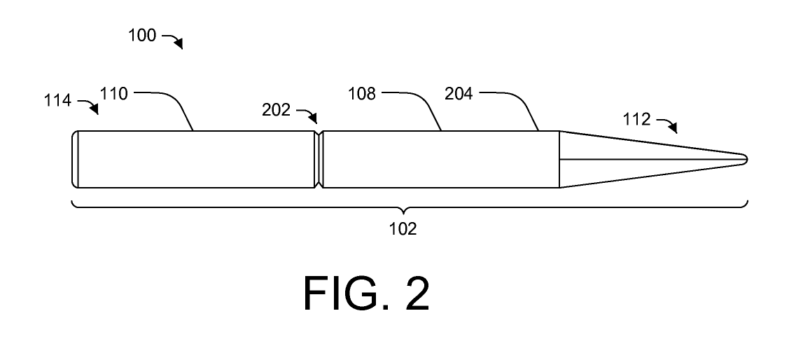

FIG. 2 is a side view of an illustrative closed disposable swab assembly 100 further illustrating the embodiment of FIG. 1. In particular, the end 112 of the first hollow portion 108 and a failure zone 202 are shown. The failure zone 202 connects the first hollow portion 108 and the second hollow portion 110, which is also displayed in FIG. 2. In this particular embodiment, the failure zone 202 comprises a notch or a reduction in wall thickness of the casing 102. However, in other embodiments, the failure zone 202 may instead or in addition comprise a weaker portion of the casing, a perforated (or partially perforated) line, a notched section, a scored section, a thin section, or any other suitable mechanism for providing a failure zone. Furthermore, in this particular embodiment the failure zone 202 is disposed on an outside surface 204 of the casing 102. However, in other embodiments, the failure zone may be disposed inside the casing 102. Depending on the particular kind of failure zone 202 chosen, the failure zone 202 may be an integral part of the casing 102 structure.

### Alternative Illustrative Disposable Swab Assembly

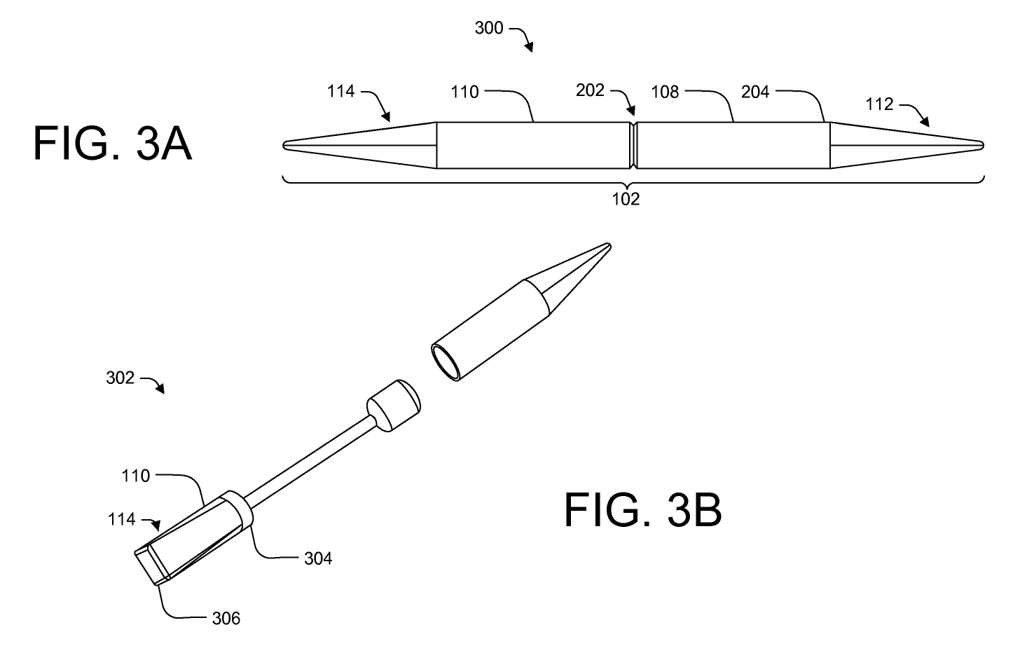

FIG. 3A is a side view of an alternative illustrative closed disposable swab assembly 300 further illustrating an embodiment in which both ends (112 and 114 respectively in the figures) of the respective hollow portions are narrowed towards their respective ends. However, as discussed above, the ends may be crimped in other embodiments depending, for example, on the manufacturing process chosen. This figure also illustrates the casing 102 and failure zone 202. As discussed previously, the failure zone 202 comprises a reduction in wall thickness of the casing 102 in this particular embodiment disposed on the outside surface 204 of the casing. However, the failure zone 202 may instead or in addition comprise any of the other configurations described elsewhere in the application.

FIG. 3B is a perspective view of an alternative illustrative opened disposable swab assembly 302 further illustrating an embodiment in which the second hollow portion 110 transitions from a circular diameter at the failure zone 202 to a square end 306 at the end 114 in this particular embodiment. In other embodiments the second hollow portion 110 may also transition to any polygon or non-linearly closed figure. For example a rectangle, triangle, hexagon, octagon, or ergonomic shape may be used.

### Illustrative Support Structure

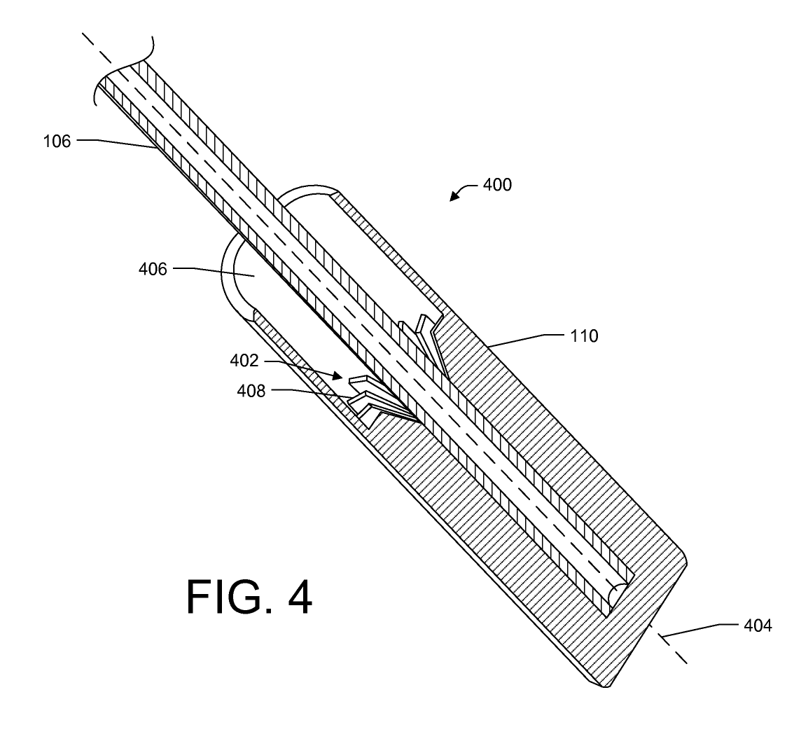

FIG. 4 is a cross-section of a perspective view of the second hollow portion 110, providing a view of an illustrative support structure 400 for engaging the stick 106. In this embodiment, the support structure 400 comprises ribs 402, lengths of which are disposed substantially parallel to a longitudinal axis 404 of the second hollow portion 110. Furthermore, the ribs 402 are coupled to a surface 406 of the inside diameter of the second hollow portion 110. Furthermore, in this embodiment the ribs 402 are coupled substantially tangent to the surface 406 of the inner diameter of the second hollow portion 110 and extending radially from the respective contact points 406 along the circumference of the stick 106 at which the ribs engage the stick 106. In this embodiment, the ribs 402 are shown to be substantially planar. However, in other embodiments, the ribs may have varying widths thereby producing ribs having surfaces that could be described as being curved. In further embodiments, the ribs 402 may not constantly be coupled with the surface 404 of the inner diameter of the second hollow portion 110 or constantly engage the stick 106 as depicted in FIG. 4, but may instead be coupled with the surface 404 or engaged with the stick 106 at a number of points.

Furthermore, edges 408 of the ribs 402 disposed away from the end 114 of the second hollow portion 110 and towards the swab may form an acute angle relative to the portion of the surface 406 to which the ribs 402 are coupled, thereby facilitating easier insertion of the swab stick 106. A second angle may be added to the edges 408, decreasing the angle relative to the surface 406 to which the ribs 402 are coupled, thereby increasing a structural integrity and strength of the ribs 402. In further embodiments, any number of additional angles may be added to the edge so that the angle between the edge and the portion of the surface 404 to which the ribs 402 are coupled progressively decreases. In other embodiments, enough angles may be added such that the equations describing the edge would no longer be linear and would become a power, exponential, or other function describing a curve.

In some embodiments, the casing 102 and the support structure 400 may be formed as an integral unit of a single material. For example, the casing 102 may be formed, using a variety of manufacturing processes, such as injection molding, blow molding, or a blow-fill-seal process. Depending on the product to be contained and the manufacturing process used, the dispenser 102 may be made of a polymer, such as polyethylene, ethyl vinyl alcohol copolymer, low-density polyethylene (LDPE), high-density polyethylene (HDPE), polypropylene (PP) or any other suitable polymer, mixture or the like that is suitable for forming the dispenser 102.

In other embodiments, the support structure 400 may be formed separately from the casing 102 and inserted into the casing 102 during an assembly process.

### Alternative Illustrative Support Structure

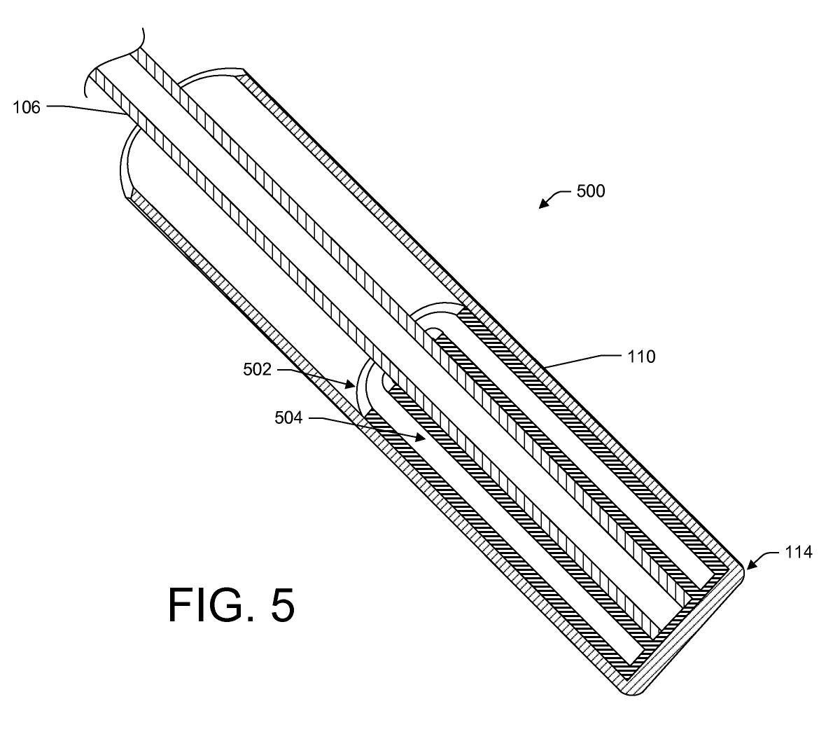

FIG. 5 is a cross-section of a perspective view of the second hollow portion 110 of an alternative illustrative support structure 500 for engaging the stick 106. In this embodiment, the support structure 500 comprises a first cylinder 502 coupled to end 114 of the second hollow portion 110 and the inside diameter of the second hollow portion 110 and a second cylinder 504 spaced from the first cylinder coupled to the end 114 of the second hollow portion 110 and engaging the stick 106.

In other embodiments, multiple cylinders may be used or the components of this embodiment of the support structure may be generally cylindrical in that the components have an average diameter. For example, a structure may replace the first or second cylinder having an inside diameter, a wall thickness, and six faces on the outside generally describing a hexagonally shaped exterior thereby being coupled with the inside diameter of the hollow portion at six points rather than continuously around the circumference of the inside diameter of the second hollow portion 110.

### Further Illustrative Embodiments of the Support Structure

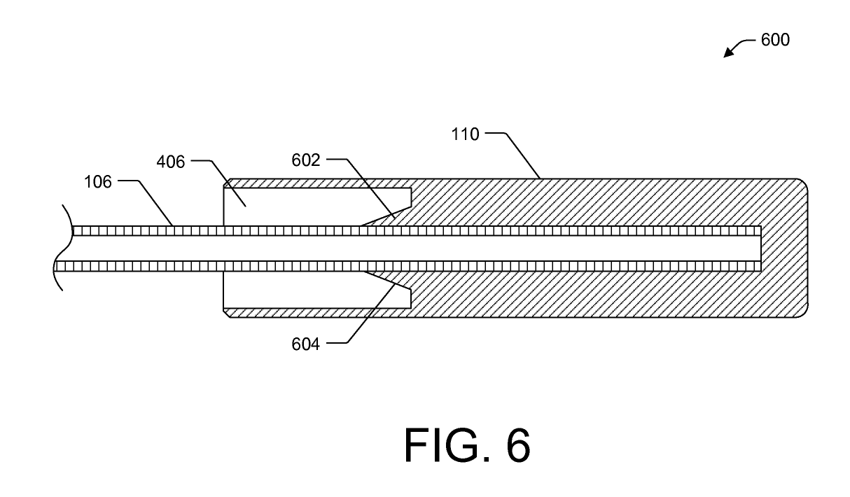

FIG. 6 is a cross-sectional side view of a further embodiment of a support structure. In the depicted embodiment, support structure 600 comprises ribs 602 similar to the ribs 402 illustrated in FIG. 4. However in this embodiment, edges 604 of the ribs 602 disposed away from the end 114 of the second hollow portion 110 and toward the swab form an obtuse angle relative to the portion of the surface 406 to which the ribs 602 are coupled.

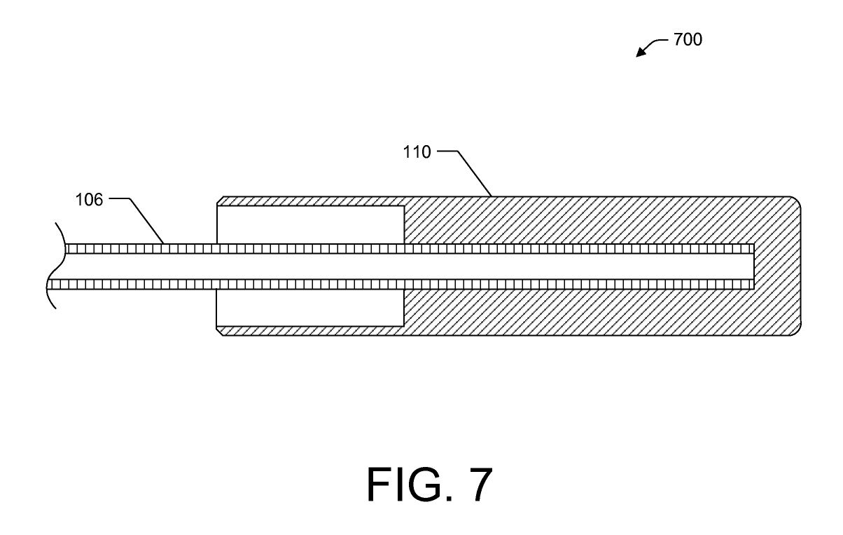

FIG. 7 is a cross-sectional side view of yet another embodiment of a support structure. In this embodiment, support structure 700 is in continual contact with the stick 106. That is, in this embodiment, the support structure 700 comprises a solid mass of a material into which the swab stick 106 is inserted. In another variation, support structure 700 may comprise ribs similar to those shown in FIG. 4, except with the rib end being normal to the length of the swab stick 106 in other embodiments. Furthermore, in other embodiments, support structure 700 may be a portion of the second hollow portion 110 that is in continual contact with the stick 106 (as shown in FIG. 7), or may be a separate insert disposed within the hollow portion 110 (not shown).

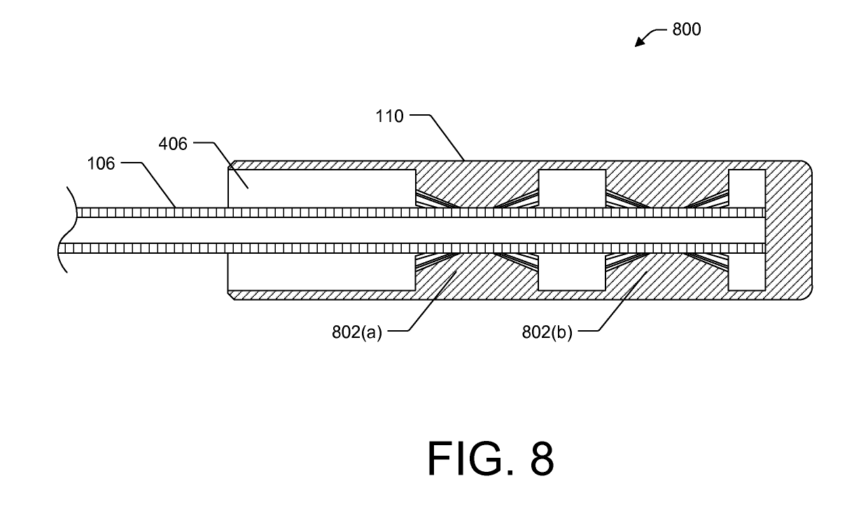

FIG. 8 is a cross-sectional side view of a further embodiment of a support structure 800. In this embodiment, support structure 800 comprises a set of ribs 802(a) and 802(b) configured to engage the stick 106 and be coupled to the surface 406 of the inside diameter of the second hollow portion 110 at two points of engagement/coupling. In other embodiments, more than two sets of ribs and more than two points of engagement/coupling may be used.

Further embodiments of the support structure may include substantially planar circular ribs having a thickness spaced from each other along the longitudinal axis of stick 106 and individually coupled to the inner circumference of the inner diameter of the second hollow portion 110 and engaging the stick 106. In this other embodiment, the ribs are disposed tangent to the longitudinal axis of the stick 106.

Any of the foregoing support structure examples may be combined or modified to arrive at other embodiments. For example, in one alternative embodiment, the distal end of the swab stick 106 opposite the swab may be disposed in a cylindrical receptacle (e.g., a shorter version of that shown in FIG. 5) and a mid section of the swab stick 106 may be supported by a rib (e.g., like that shown at 802a in FIG. 8). These and other modifications and variations are possible.

### Illustrative Applicators

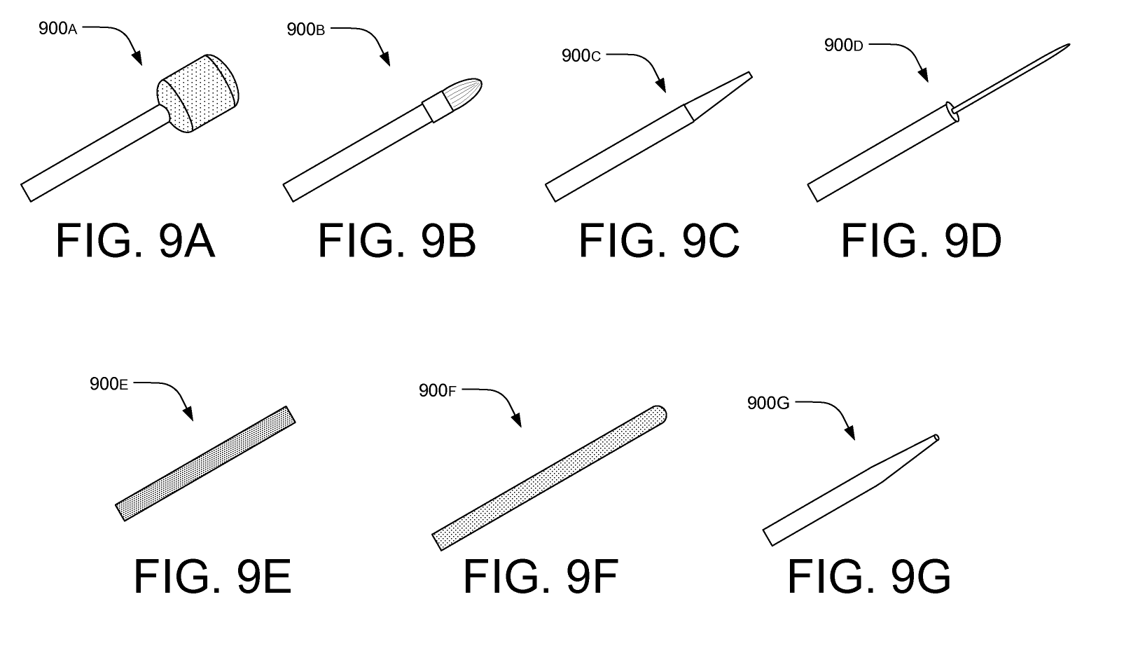

FIGS. 9A, 9B, 9C, 9D, 9E, 9F, and 9G illustrate several alternative applicators that may be used with the applicator assemblies described herein. Any of the applicators described in this section may be substituted for the swab applicators in the applicator assemblies described above.

FIG. 9A illustrates an example swab applicator 900A comprising a natural or synthetic sponge material. FIG. 9B illustrates an example brush applicator 900B. The brush applicator may include natural or synthetic hairs or bristles. FIG. 9C illustrates an example pick applicator 900C. FIG. 9D illustrates an example needle applicator 900D. FIG. 9E illustrates an example stick applicator 900E. FIG. 9F illustrates an example tongue depressor applicator 900F. FIG. 9G illustrates an example pipette applicator 900G.

The size, shape and configuration of the applicators 900A-900G may be configured depending on, for example, a product to be applied with the pick applicator and/or a surface to which the product is to be applied. Moreover, any of the applicators 900A-900G may be coated, impregnated, saturated, filled, or may otherwise include a product to be applied to a surface. Products may include medicinal products, personal care products, cosmetic products, adhesives, or any other product that can be applied using the aforementioned applicators. In some examples, such products may be in the form of liquids, gels, creams, powders, emulsions, or solids. In some examples, the applicator may actually be formed of the product that is to be applied (e.g., a pick applicator formed of solid, soluble oral care material).

### Methods for Opening the Illustrative Disposable Swab Assembly

Illustrative closed disposable swab assembly 200 or alternative illustrative closed disposable swab assembly 300 may be opened using the failure zone 202 by a variety of methods. For example, users may bend the casing in order to apply force to the failure zone, thereby breaking the casing 102 at the failure zone 202 and exposing the swab. Users may instead or additionally apply torsional, tensile, shear, or conjoint forces to the swab assembly 200 or 300. Examples of application of such forces may include but are not limited to pushing together or pulling apart the first hollow portion 108 and the second hollow portion 110, grasping the first hollow portion 108 and the second hollow portion 110 and twisting, squeezing the failure zone 202, or using a cutting device at the failure zone 202.

### Example Process for Manufacturing a Disposable Swab Assembly

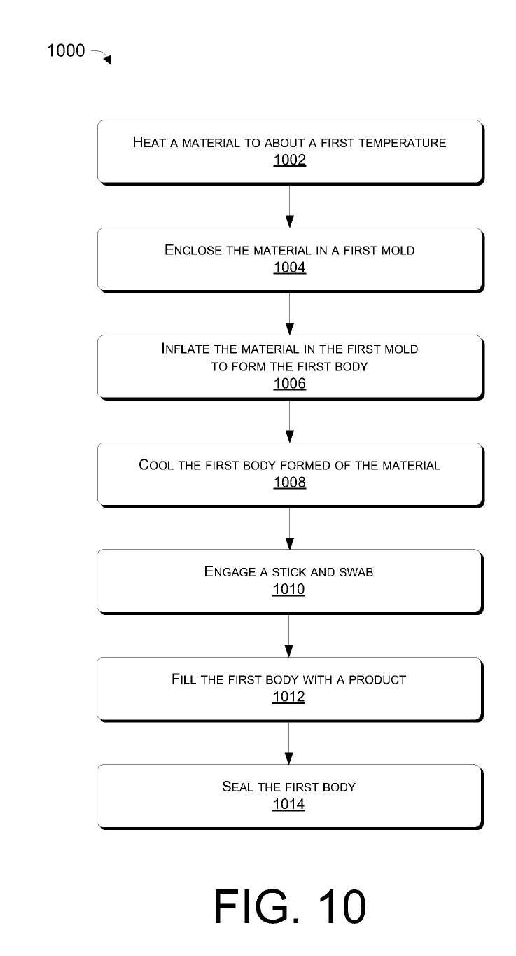

FIG. 10 illustrates an example process 1000 for manufacturing a disposable swab assembly (e.g., disposable swab assembly 100) based at least in part on material characteristics of the particular disposable swab assembly. For instance, this process may be performed to manufacture a disposable swab assembly comprising a unit formed of a single material. For example, the disposable swab assembly and each of the disposable swab assembly's constituents may be formed of a polymer, such as polyethylene, polypropylene, ethyl vinyl alcohol copolymer or any other suitable polymer, mixture or the like that is suitable for forming the disposable swab assembly. Suitability of materials may be based on the materials' ability to be molded, its permeability, its resistance to reaction/degradation in proximity to certain products, and the like. In some instances, the process may be performed at a manufacturing facility prior to the shipping of the disposable swab assembly. Additionally, the manufacturing facility may be capable of aseptic processing for producing non-disposable swab assemblies. Additionally, this process may apply to manufacturing any type of swab assembly formed of any other suitable materials capable of being manufactured by injection molding, blow molding, blow-fill-seal processing, or any other suitable manufacturing process.

Process 1000 includes an operation 1002, which represents heating a material (e.g., polyethylene, polypropylene, ethyl vinyl alcohol copolymer) to a first temperature (e.g., about 130 degrees Celsius in the case of one particular embodiment). Next, process 1000 proceeds to operation 1004, which represents enclosing the material in a first mold. The first mold comprising a shape to form a first body. The mold includes cavities and/or protrusions to form a first hollow portion (e.g., first hollow portion 108) frangibly coupled to a second hollow portion (e.g., second hollow portion 110) which is included in the mold. The mold is further configured to provide a failure zone (e.g., failure zone 202), the failure zone being weaker relative to the remainder of disposable swab assembly, such that the failure zone is configured to break at the failure zone upon application of a predetermined force. Alternatively, in other embodiments, the failure zone could be formed as a subsequent finishing process (e.g., scoring) rather than as part of the molding process. The mold is further configured to provide a support structure (e.g., support structures 400 and 500). However in the illustrated process the swab stick is formed separately and subsequently inserted in the support structure 400 (or 500, 600, 700, or 800 depending on the particular embodiment). In some embodiments, a stick (e.g., stick 106) may also be formed by the mold. Process 1000 continues to operation 1006, where, a mandrel is used to inflate the material in the first mold to form the first body. Operation 1006 is followed by operation 1008 where the first body formed of the material is cooled (e.g., to about 50 degrees Celsius). Following operation 1008, at operation 1010 a stick and a swab (e.g., stick 106 and swab 104) may be attached and engaged with the support structure. Process 1000 continues with operation 1012, and subsequent to the cooling of the first body formed of the material, a mandrel is used to fill the first body with a product (e.g., a cosmetic, oral care product, medicine, or other product). In other embodiments, the swab may be inserted with the product previously applied to the swab. After operation 1012, process 1000 continues with operation 1014, where a second mold is used to seal the first body thereby forming the disposable swab assembly with a swab contained.

### CONCLUSION

Although embodiments have been described in language specific to structural features and/or methodological acts, the embodiments are not necessarily limited to the specific features or acts described. Rather, the specific features and acts are disclosed as illustrative forms of implementing the embodiments. For example, while embodiments are described having certain shapes, sizes, and configurations, these shapes, sizes, and configurations are merely illustrative. Also, while one example manufacturing process is described, disposable swab assemblies according to this disclosure may be made using any other suitable manufacturing process.

What is claimed is:

1. An applicator assembly comprising:
a casing, the casing comprising:
a first hollow portion having an inside diameter;
a second hollow portion having an inside diameter substantially the same as the inside diameter of the first hollow portion;
a stick contained within the first and second hollow portions of the casing, having an applicator disposed at one end of the stick; and
a support structure coupled to the casing and engaging the stick.

2. The applicator assembly of claim 1, further comprising a failure zone for opening the applicator assembly.

3. The applicator assembly of claim 2, the failure zone comprising a reduction in wall thickness of the casing or a weaker portion of the casing, and wherein the failure zone connects the first and second hollow portions.

4. The applicator assembly of claim 1, the end of the first hollow portion being crimped.

5. The applicator assembly of claim 1, the support structure comprising ribs disposed substantially parallel to a portion of the length of the second hollow portion and coupled to the inside diameter of the second hollow portion.

6. The applicator assembly of claim 5, the ribs being coupled substantially tangent to a surface of the inner diameter of the second hollow portion and extending radially from respective points at which the ribs engage the stick.

7. The applicator assembly of claim 1, the support structure comprising:
a first cylinder, the first cylinder being coupled to the end and the inside diameter of the second hollow portion; and
a second cylinder spaced from the first cylinder, the second cylinder being coupled to the end of the second hollow portion and engaging the stick.

8. The applicator assembly of claim 1, the casing being of a generally cylindrical shape.

9. The applicator assembly of claim 1, the swab assembly being injection molded, blow molded, injection-blow molded, or formed by a blow-fill-seal process.

10. The applicator assembly of claim 1, further comprising a product housed within the casing.

11. The applicator assembly of claim 1, the applicator comprising a swab applicator.

12. The applicator assembly of claim 1, the applicator comprising a brush, a pick, a needle, a stick, a tongue depressor, a pipette, or a combination of any of the foregoing.

13. A swab assembly comprising:
a casing; and
an elongated swab applicator having a swab end and a stick end, the swab applicator being coupled to a support structure, the support structure being interposed between the casing and the stick end of the elongated swab applicator to support the swab applicator relative to the casing.

14. The swab assembly of claim 13, the casing comprising first and second enclosure portions of substantially the same inner diameter.

15. The swab assembly of claim 14, further comprising a failure zone, the failure zone being a weaker region of the swab assembly in comparison to the first and second enclosure portions and wherein the first and second enclosure portions are connected by their respective connections to the failure zone.

16. The swab assembly of claim 14, the ends of the first and second enclosure portions being crimped.

17. The swab assembly of claim 14, the support structure comprising ribs extending radially from the stick end of the swab applicator and being disposed substantially parallel to a portion of a length of the casing and coupled to an inside diameter of the second enclosure portion.

18. The support structure of claim 17, the ribs being coupled substantially tangent to the surface of the inner diameter of the casing and extending radially from the respective points at which the ribs engage the stick.

19. The swab assembly of claim 14, the support structure comprising:
a first cylinder coupled to the end and the inside diameter of the second enclosure portion; and
a second cylinder spaced from the first coupled to the end of the second enclosure portion and engaging the stick.

20. The swab assembly of claim 13, the swab assembly being injection molded, blow molded, injection-blow molded, or formed by a blow-fill-seal process.

21. The swab assembly of claim 13, further comprising a product housed within the casing.

22. The swab assembly of claim 13, the casing being of a generally cylindrical shape.

23. A method of manufacturing an applicator assembly comprising:
forming a casing having a substantially uniform diameter;
forming a support structure within and coupled to the casing; and
inserting an applicator inside the casing and into the support structure, such that the applicator is supported by the support structure.

24. The method of claim 23, wherein forming the support structure and the casing comprises:
heating a material to a first temperature;
enclosing the material in a mold, the mold comprising a shape to form a body;
inflating the material in the mold to form the body; and
cooling the body formed of the material.

25. The method of claim 23, further comprising inserting a product into the casing and sealing the casing.
